(19) United States
(12) Patent Application Publication
(10) Pub. No.: US 2004/0030166 A1
(43) Pub. Date: Feb. 12, 2004

(54) Methods for treating deodorizer distillate

(76) Inventors: Dick Copeland, Omaha, NE (US); W. Maurice Belcher, Omaha, NE (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/100,443

(22) Filed: Mar. 18, 2002

(51) Int. Cl. C07J 53/00; C07D 311/76; C11B 1/00

(52) U.S. Cl. 552/545; 549/413; 554/9

(57) ABSTRACT

This invention relates to methods for treating distillates obtained during the process of deodorizing various oils. More particularly, this invention relates to methods for recovering fatty acids, tocopherols, and sterols from a distillate obtained from the deodorizing of various oils.

### FIELD OF THE INVENTION

This invention relates to methods for treating distillates obtained during the process of deodorizing various oils. More particularly, this invention relates to methods for recovering fatty acids, tocopherols, and sterols from a distillate obtained from the deodorizing of various oils.

### BACKGROUND OF THE INVENTION

Oils derived from plants and animals are valuable sources of fatty acids, tocopherols, and sterols. During the process of refining such oils, however, significant amounts of these components, especially the tocopherols and sterols, are lost to various intermediate byproducts and waste streams, which include acidulated soapstocks, deodorizer distillates, or both, depending on the refining method selected. Accordingly, numerous methods have been proposed for recovering fatty acids, tocopherols, and sterols from various refining intermediates, including deodorizer distillates that are obtained as byproducts of a high-temperature distillation step (commonly termed deodorization) during the production of oils and fats.

Deodorization is usually the final step in producing edible oils and fats from plant and animal sources. Vegetable oils such as soybean oil typically contain volatile impurities that can impart objectionable odor and taste. These volatile compounds generally must be removed to produce edible oils. Deodorization generally involves a steam stripping process wherein steam is contacted with oil in a distillation apparatus operating at low pressure and a temperature sufficient to vaporize objectionable volatile impurities at the operating pressure. This process, commonly known as vacuum-steam deodorization, relies upon volatility differences between the oil and the objectionable impurities to strip the relatively more volatile objectionable impurities from the relatively less volatile oil. In a typical vacuum-steam deodorizing process, vegetable oil is introduced into a distillation apparatus having a plurality of vertically spaced trays, commonly termed stripping trays. Within each stripping tray, steam injected into the vegetable oil enhances removal of objectionable volatile impurities. The combined steam and entrained distillation vapors are usually collected and condensed to form a distillate that can be disposed of or processed further to recover valuable materials.

The major constituents of deodorizer distillates are fatty acids, tocopherols, and sterols, which are present in various relative amounts depending on the oil source and the refining steps the oil is subjected to prior to deodorization. Deodorizer distillate itself has a certain commercial value. However, greater value can be realized when deodorizer distillate is split into a fatty acid-enriched fraction and a fraction enriched in sterols and tocopherols. Even greater value can be realized when the fraction enriched in sterols and tocopherols is subsequently split into a sterol-enriched fraction and a tocopherol-enriched fraction.

Fatty acids isolated from deodorizer distillates are utilized in several nonfood applications and are particularly useful as fluidizing agents for lecithin. Such fatty acids also can be utilized as precursors in a wide variety of molecular synthesis schemes. Typically, the fatty acid portion of deodorizer distillate comprises C₁₀-C₂₂ saturated and unsaturated fatty acids. Soybean deodorizer distillate in particular contains about 50 percent by weight fatty acids.

Deodorizer distillates also contain sterols, which are valuable precursors in the production of hormones. Stigmasterol is used in manufacturing progesterone and corticoids. Sitosterol is used to produce estrogens, contraceptives, diuretics, and male hormones. Soybean deodorizer distillate in particular contains from about 10 to about 18 percent by weight total sterols, of which about 50% is sitosterol, about 20% is stigmasterol, about 20% is campesterol, and about 10% is other minor sterols.

The final major component of deodorizer distillates is tocopherol. Tocopherols are valuable natural antioxidants that help prevent oxidation and spoilage. Tocopherols are also utilized in the production of Vitamin E. Distillates obtained from soybean oil deodorization generally contain a mixture of α, β, γ, and δ tocopherol isomers in a ratio of about 15:5:30:50. Alpha tocopherol has the most powerful biological Vitamin E activity. The other tocopherols have weaker Vitamin E activity but stronger antioxidant activity. If maximum Vitamin E activity is desired, non-alpha tocopherols can be converted into the alpha form by well-known techniques, such as methylation.

In the past, recovering tocopherols and sterols from deodorizer distillates and related mixtures has proved complicated and expensive. One difficulty associated with isolating one or more distillate fractions enriched in fatty acids, tocopherols, and/or sterols from deodorizer distillates is that the boiling points of sterols and tocopherols are roughly in the same range. Another difficulty is that deodorizer distillate can undergo thermal degradation if it is processed for extended periods at the temperatures at which sterols and tocopherols vaporize, such temperature conditions which can cause fatty acids to convert into undesirable trans isomeric forms.

Numerous methods have been proposed for treating deodorizer distillates to isolate and recover one or more components. In many of these methods, a first essential process step involves subjecting the fatty acids to an esterification or saponification reaction. For example, U.S. Pat. No. 3,153,055 teaches a process for isolating sterols and tocopherols from deodorizer distillate by esterifying the fatty acids with a monohydric alcohol under strongly acidic conditions. The sterols and tocopherols are then fractionally extracted from the esterification product mixture with a combination of polar and nonpolar solvents.

In an alternative esterification method, U.S. Pat. No. 5,487,817 teaches esterifying the sterols with the fatty acids and then distilling the resulting mixture to obtain a residue containing sterol esters and a distillate containing tocopherols. Sterols are then isolated from the residue by subjecting the sterol esters to cleavage under acidic conditions.

U.S. Pat. No. 2,349,270 discloses that deodorizer distillate can be treated with lime soap to saponify the fatty acids, followed by extraction of the unsaponifiable fraction (tocopherols and sterols) with acetone, in which the saponification products are insoluble. The extract is then washed and concentrated, as for example by solvent distillation, and then cooled to crystallize sterols which are removed by filtration, leaving a high purity tocopherol fraction. The fatty acid soaps formed by the process can be acidulated and converted into free fatty acids.

Extractive separation methods also have been employed in treating deodorizer distillates to isolate one or more components. For example, U.S. Pat. No. 5,138,075 describes a method for recovering tocopherols from a deodorized distillate which comprises contacting the distillate with liquid water at elevated temperature and pressure, thereby producing a raffinate phase stream having a relatively high concentration of tocopherols and an extract phase stream having a relatively high concentration of fatty acids. The raffinate stream and the extract stream are then cooled to a temperature at which the organic components thereof are immiscible with liquid water, whereupon removal of water produces a tocopherol-enriched fraction and a fatty acid-enriched fraction, respectively.

None of the above methods for isolating one or more components from a deodorizer distillate has proved satisfactory, however. Methods employing an esterification step or saponification step introduce processing complexity and require later processing steps that often involve use of strong mineral acids in order to convert the respective esters or soaps into free sterols and free fatty acids. Mineral acids can be dangerous in handling and can induce discoloration or other degradation of distillate components. Methods requiring extractive steps are expensive and create the potential for contamination by residual solvent.

Previously known methods for isolating one or more components from a deodorizer distillate generally have required lengthy and costly processing steps. Consequently, further improvements in methods for treating deodorizer distillates have been sought. The present invention relates to improved processes having advantages over those previously disclosed. The methods of the invention produce a fatty acid-enriched condensate directly and simply from a liquid distillate. The methods of the invention also produce a distillate fraction enriched in sterols and tocopherols, which can be treated further by various methods to isolate a sterol fraction and a tocopherol fraction.

### SUMMARY OF THE INVENTION

One aspect of the present invention relates to methods for isolating one or more components from liquid distillates obtained from the deodorization of various oils.

Another aspect of the present invention relates to methods for producing fatty acid-enriched mixtures from liquid distillates obtained from the deodorization of various oils.

Yet another aspect of the invention relates to methods for producing mixtures enriched in sterols and tocopherols from liquid distillates obtained from the deodorization of various oils.

A further aspect of the invention relates to methods for producing mixtures enriched in sterols from distillate fractions enriched in sterols and tocopherols.

A still further aspect of the invention relates to methods for producing mixtures enriched in tocopherols from distillate fractions enriched in sterols and tocopherols.

One embodiment of the invention is a process for isolating components from a distillate that comprises the steps of introducing a distillate comprising sterols, tocopherols, and fatty acids into a heating zone operating at a pressure of less than about 10 mm Hg and at a temperature of less than about 480° F.; vaporizing a substantial fraction of the fatty acids to produce a vapor phase enriched in fatty acids, leaving a remaining fraction of distillate enriched in sterols and tocopherols; and cooling the vapor phase to produce a condensate enriched in fatty acids.

Another embodiment of the invention is a process for isolating components from a distillate that comprises the steps of preheating a distillate comprising sterols, tocopherols, and fatty acids; introducing the preheated distillate into a heating zone operating at a pressure of less than about 10 mm Hg and at a temperature of less than about 480° F.; vaporizing a substantial fraction of the fatty acids to produce a vapor phase enriched in fatty acids, leaving a remaining fraction of distillate enriched in sterols and tocopherols; and cooling the vapor phase to produce a condensate enriched in fatty acids.

Yet another embodiment of the invention is a process for isolating components from a distillate that comprises the steps of preheating a distillate comprising sterols, tocopherols, and fatty acids; introducing the preheated distillate into a heating zone operating at a pressure of less than about 10 mm Hg and at a temperature of less than about 480° F.; contacting the preheated distillate with a stripping gas; vaporizing a substantial fraction of the fatty acids to produce a vapor phase enriched in fatty acids, leaving a remaining fraction of distillate enriched in sterols and tocopherols; and cooling the vapor phase to produce a condensate enriched in fatty acids.

Still another embodiment of the invention is a process for isolating components from a distillate that comprises the steps of introducing a distillate comprising sterols, tocopherols, and fatty acids into a heating zone operating at a pressure of less than about 10 mm Hg and at a temperature of less than about 480° F.; vaporizing a substantial fraction of the fatty acids to produce a first vapor phase enriched in fatty acids, leaving a remaining fraction of distillate enriched in sterols and tocopherols; cooling the remaining fraction of distillate; combining acetone and the remaining fraction of distillate to produce a precipitate enriched in sterols and a solvent phase enriched in tocopherols; and separating the precipitate and the solvent phase.

A further embodiment of the invention is a process for isolating components from a distillate that comprises the steps of preheating a distillate comprising sterols, tocopherols, and fatty acids; introducing the preheated distillate into a heating zone operating at a pressure of less than about 10 mm Hg and at a temperature of less than about 480° F.; vaporizing a substantial fraction of the fatty acids to produce a first vapor phase enriched in fatty acids, leaving a remaining fraction of distillate enriched in sterols and tocopherols; cooling the remaining fraction of distillate; combining acetone and the remaining fraction of distillate to produce a precipitate enriched in sterols and a solvent phase enriched in tocopherols; and separating the precipitate and the solvent phase.

A still further embodiment of the invention is a process for isolating components from a distillate that comprises the steps of preheating a distillate comprising sterols, tocopherols, and fatty acids; introducing the preheated distillate into a heating zone operating at a pressure of less than about 10 mm Hg and at a temperature of less than about 480° F.; contacting the preheated distillate with a stripping gas; vaporizing a substantial fraction of the fatty acids to produce a first vapor phase enriched in fatty acids, leaving a remaining fraction of distillate enriched in sterols and tocopherols; cooling the remaining fraction of distillate; combining acetone and the remaining fraction of distillate to produce a precipitate enriched in sterols and a solvent phase enriched in tocopherols; and separating the precipitate and the solvent phase.

A still further embodiment of the invention is a process for isolating components from a distillate that comprises the steps of preheating a distillate comprising sterols, tocopherols, and fatty acids; introducing the preheated distillate into a heating zone operating at a pressure of less than about 10 mm Hg and at a temperature of less than about 480° F.; contacting the preheated distillate with a stripping gas; vaporizing a substantial fraction of the fatty acids to produce a first vapor phase enriched in fatty acids, leaving a remaining fraction of distillate enriched in sterols and tocopherols; cooling the first vapor phase to produce a condensate enriched in fatty acids; cooling the remaining fraction of distillate; combining acetone and the remaining fraction of distillate to produce a precipitate enriched in sterols and a solvent phase enriched in tocopherols; separating the precipitate and the solvent phase; and vaporizing a substantial fraction of the acetone from the solvent phase to produce a second vapor phase enriched in acetone, leaving a residue enriched in tocopherols.

An additional embodiment of the invention is a process for isolating components from a distillate that comprises the steps of introducing a distillate comprising sterols, tocopherols, and fatty acids into a first heating zone operating at a pressure of less than about 10 mm Hg and at a temperature of less than about 480° F.; vaporizing a substantial fraction of the fatty acids to produce a first vapor phase enriched in fatty acids, leaving a first remaining fraction of distillate enriched in sterols and tocopherols; introducing the first remaining fraction of distillate into a second heating zone operating at a pressure of less than about 10 mm Hg and at a temperature of from about 450 to about 525° F.; and vaporizing a substantial fraction of the tocopherols to produce a second vapor phase, leaving a second remaining fraction of distillate enriched in sterols.

A further additional embodiment of the invention is a process for isolating components from a distillate that comprises the steps of preheating a distillate comprising sterols, tocopherols, and fatty acids; introducing the preheated distillate into a first heating zone operating at a pressure of less than about 10 mm Hg and at a temperature of less than is about 480° F.; vaporizing a substantial fraction of the fatty acids to produce a first vapor phase enriched in fatty acids, leaving a first remaining fraction of distillate enriched in sterols and tocopherols; introducing the first remaining fraction of distillate into a second heating zone operating at a pressure of less than about 10 mm Hg and at a temperature of from about 450 to about 525° F.; and vaporizing a substantial fraction of the tocopherols to produce a second vapor phase, leaving a second remaining fraction of distillate enriched in sterols.

An even further additional embodiment of the invention is a process for isolating components from a distillate that comprises the steps of preheating a distillate comprising sterols, tocopherols, and fatty acids; introducing the preheated distillate into a first heating zone operating at a pressure of less than about 10 mm Hg and at a temperature of less than about 480° F.; contacting the preheated distillate with a stripping gas; vaporizing a substantial fraction of the fatty acids to produce a first vapor phase enriched in fatty acids, leaving a first remaining fraction of distillate enriched in sterols and tocopherols; introducing the first remaining fraction of distillate into a second heating zone operating at a pressure of less than about 10 mm Hg and at a temperature of from about 450 to about 525° F.; and vaporizing a substantial fraction of the tocopherols to produce a second vapor phase, leaving a second remaining fraction of distillate enriched in sterols.

A still further additional embodiment of the invention is a process for isolating components from a distillate that comprises the steps of preheating a distillate comprising sterols, tocopherols, and fatty acids; introducing the preheated distillate into a first heating zone operating at a pressure of less than about 10 mm Hg and at a temperature of less than about 480° F.; contacting the preheated distillate with a stripping gas; vaporizing a substantial fraction of the fatty acids to produce a first vapor phase enriched in fatty acids, leaving a first remaining fraction of distillate enriched in sterols and tocopherols; introducing the first remaining fraction of distillate into a second heating zone operating at a pressure of less than about 10 mm Hg and at a temperature of from about 450 to about 525° F.; contacting the first remaining distillate with a stripping gas; and vaporizing a substantial fraction of the tocopherols to produce a second vapor phase, leaving a second remaining fraction of distillate enriched in sterols.

A yet further additional embodiment of the invention is a process for isolating components from a distillate that comprises the steps of preheating a distillate comprising sterols, tocopherols, and fatty acids; introducing the preheated distillate into a first heating zone operating at a pressure of less than about 10 mm Hg and at a temperature of less than about 480° F.; contacting the preheated distillate with a stripping gas; vaporizing a substantial fraction of the fatty acids to produce a first vapor phase enriched in fatty acids, leaving a first remaining fraction of distillate enriched in sterols and tocopherols; cooling the first vapor phase to produce a condensate enriched in fatty acids; introducing the first remaining fraction of distillate into a second heating zone operating at a pressure of less than about 10 mm Hg and at a temperature of from about 450 to about 525° F.; contacting the first remaining distillate with a stripping gas; and vaporizing a substantial fraction of the tocopherols to produce a second vapor phase, leaving a second remaining fraction of distillate enriched in sterols.

An additional further embodiment of the invention is a process for isolating components from a distillate that comprises the steps of preheating a distillate comprising sterols, tocopherols, and fatty acids; introducing the preheated distillate into a first heating zone operating at a pressure of less than about 10 mm Hg and at a temperature of less than about 450° F.; contacting the preheated distillate with a stripping gas; vaporizing a substantial fraction of the fatty acids to produce a first vapor phase enriched in fatty acids, leaving a first remaining fraction of distillate enriched in sterols and tocopherols; cooling the first vapor phase to produce a condensate enriched in fatty acids; introducing the first remaining fraction of distillate into a second heating zone operating at a pressure of less than about 10 mm Hg and at a temperature of from about 450 to about 525° F.; contacting the first remaining distillate with a stripping gas; vaporizing a substantial fraction of the tocopherols to produce a second vapor phase, leaving a second remaining fraction of distillate enriched in sterols; and cooling the second vapor phase to produce a second condensate enriched in tocopherols.

These and other aspects of the invention will become apparent in light of the detailed description below.

As used herein, the term “comprising” means including, but not limited to, whatever follows the word “comprising.” Thus, use of the term comprising indicates that listed elements are required or mandatory, but that other elements are optional and may be present.

As used herein, the term “non-condensible inert gas” means any one or mixture of inert gases that do not condense at the operating temperature and pressure. Non-condensible inert gases include but are not limited to nitrogen, carbon dioxide, argon, helium, hydrogen, and mixtures thereof.

As used herein, the term “steam-free” means that steam does not come into direct contact with oil or vaporized distillate. However, steam may be utilized to supply heat indirectly, as by use of a heat exchanger.

As used herein, the term “edible oil” means any one or mixture of oils and/or fats derived from vegetable and/or animal sources. The term “vegetable” includes but is not limited to soybean, corn, cottonseed, palm, peanut, rapeseed, safflower, sunflower, sesame, rice bran, coconut, canola, and mixtures thereof. The term “animal” includes but is not limited to fish, mammal, reptile, and mixtures thereof.

### BRIEF DESCRIPTION OF THE DRAWINGS

FIG. 1 illustrates one process suitable for carrying out the methods of the present invention.

FIG. 2 illustrates another process suitable for carrying out the methods of the present invention.

FIG. 3 illustrates yet another process suitable for carrying out the methods of the present invention.

### DESCRIPTION OF A PREFERRED EMBODIMENT

All methods of the invention can be conducted as batch, semi-continuous, or continuous processes. The improved processes of the invention serve to isolate the components of distillates obtained from the deodorization of various oils. Many such distillates are suitable for use in the invention, including but not limited to those obtained from the deodorization of soybean oil, corn oil, cottonseed oil, palm oil, peanut oil, rapeseed oil, safflower oil, sunflower seed oil, sesame seed oil, rice bran oil, coconut oil, canola oil, and mixtures thereof. A particularly suitable distillate is soybean deodorizer distillate.

The composition of deodorizer distillates will vary depending upon the oil type and pre-deodorization refining history. Distillate obtained from the deodorization of alkali-refined soybean oil generally contains about 50 percent by weight fatty acids, about 15 percent by weight tocopherols, and about 18 percent by weight sterols. Distillate resulting from the deodorization of physically refined soybean oil usually comprises about 70 percent by weight fatty acids, about 9 percent by weight tocopherols, and about 11 percent by weight sterols. Distillate obtained from the deodorization of soybean oil refined via an organic acid refining process, as disclosed in U.S. Pat. No. 6,172,248, herein incorporated by reference, typically contains about 55 percent by weight fatty acids, about 12 percent by weight tocopherols, and about 14 percent by weight sterols. Any of these deodorizer distillates, concentrated forms of such distillates, or mixtures thereof, are suitable for use in the present invention.

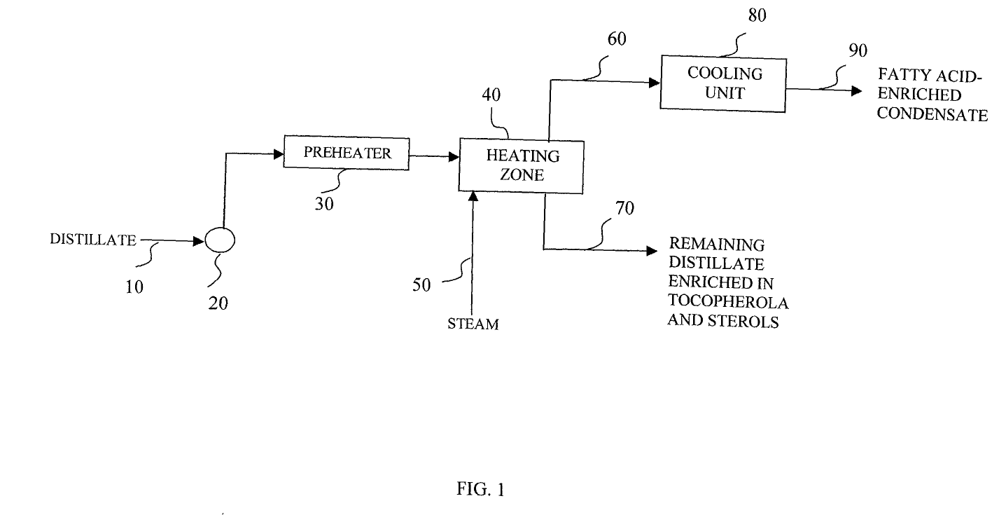

FIG. 1 illustrates one process suitable for carrying out the methods of the invention. One of ordinary skill understands that the FIGS. 1, 2, and 3 may omit a detailed showing of certain equipment, instrumentation, valving, etc., which would be used in practicing the methods of the invention, as such would be readily apparent to those skilled in distillate treatment and related processing arts. As illustrated in FIG. 1, one method of the invention for isolating components from deodorizer distillates generally entails introducing a distillate 10 comprising sterols, tocopherols, and fatty acids into a heating zone 40 operating at a pressure of less than about 10 mm Hg and at a temperature of less than about 480° F.

Heating zone 40 can comprise any equipment of sufficient volume and capable of operating at reduced pressure and elevated temperature. Preferably, heating zone 40 comprises a flash tank. Reduced pressure can be generated by any convenient source. Steam jet ejector systems are commonly employed. Also suitable is use of one or more non-steam vacuum sources, such as vacuum pumps, alone or in combination with steam jet ejector systems. Exemplary but non-limiting vacuum pumps include multistage centrifugal pumps, water- or oil-sealed rotary pumps, liquid ring vacuum pumps, or dry-vacuum reciprocating pumps. Most preferably, reduced pressure is generated by a Nash-Kinema three-stage vacuum system or a two-stage vacuum system plus a vacuum pump. With a three-stage ejector system, the usual vacuum generated in heating zone 40 will be less than about 10 mm Hg. Preferably, heating zone 40 operates at a pressure of less than about 6 mm Hg. Most preferably, heating zone 40 operates at a pressure of less than about 4 mm Hg.

Optionally, but preferably, the distillate 10 passes through a preheater 30 before being introduced into heating zone 40. Preferably, the distillate 10 is preheated to a temperature near to the operating temperature of heating zone 40. The distillate 10 can be preheated either directly, as by mixing with a separate stream of heated distillate, or indirectly, as by a convenient means such as a heat exchanger.

Within heating zone 40, a substantial fraction of the fatty acid content of distillate 10 vaporizes, producing a vapor phase 60 enriched in fatty acids and leaving a remaining fraction of distillate 70 enriched in sterols and tocopherols. To minimize the risk of thermal degradation that can occur at high processing temperatures, the distillate 10 remains in heating zone 40 for a time of less than about 60 minutes, and preferably less than about 30 minutes. Optionally, but preferably, the distillate 10 is contacted with a stripping gas to accelerate vaporization and/or removal of vaporized fatty acids. Steam is commonly employed as stripping gas. Other suitable stripping gases include but are not limited to non-condensible inert gases.

The usage rate of stripping gas will vary based on the type and flow rate of distillate, the distillate pre-deodorization history, and the dimensions of the heating zone(s). When the stripping gas is steam, it is generally used in an amount of from about 0.1 to about 5 percent by weight of distillate when the operating pressure is less than about 5 mm Hg. When the stripping gas is a non-condensible inert gas, it is preferably nitrogen that is substantially water-free and has a purity of greater than about 98 percent. A suitable nitrogen source includes but is not limited to a Praxair PSA Nitrogen System, available from Praxair Technology, Inc., Danbury, Conn. When the stripping gas is nitrogen, it is generally introduced at a rate of from about 0.1 to about 10 liters per minute when the operating pressure is less than about 5 mm Hg. More preferably, nitrogen is introduced at a rate of from about 0.5 to about 3 liters per minute, which equates generally to a rate of from about 0.2 to about 20 pounds per hundred pounds of distillate.

Heating zone 40 operates at a temperature less than the boiling point of tocopherols and sterols at the operating temperature but greater than the boiling point of fatty acids at the operating pressure. Table 1 indicates the boiling point of tocopherols and sterols at several reduced pressures.

TABLE 1

| | Tocopherols | | |
|---|---|---|---|
| Pressure | boiling | Sterols | Fatty Acids |
| (mm Hg) | point (° F.) | boiling point (° F.) | boiling point (° F.) |
| 1 | 444 | 464 | 334 |
| 2 | 468 | 473 | 354 |
| 3 | 486 | 500 | 370 |
| 4 | 500 | 518 | 380 |

Generally, heating zone 40 operates at a temperature of from about 375 to about 480° F. Preferably, heating zone 40 operates at a temperature of from about 400 to about 465° F. Most preferably, heating zone 40 operates at a temperature of from about 425 to about 450° F.

The vapor phase 60 passes through a cooling unit 130 to produce a condensate 140 enriched in fatty acids. The vapor phase 60 can be cooled either directly, as by mixing with a separate stream of cooled condensate enriched in fatty acids, or indirectly, as by a convenient means such as a heat exchanger. The condensate 140 enriched in fatty acids and the remaining fraction of distillate 70 enriched in sterols and tocopherols can be individually collected and profitably sold or further processed. Generally, condensate 140 comprises greater than about 70 percent by weight fatty acids. Generally, the remaining fraction of distillate 70 comprises at least about 20 percent by weight sterols and at least about 20 percent by weight tocopherols.

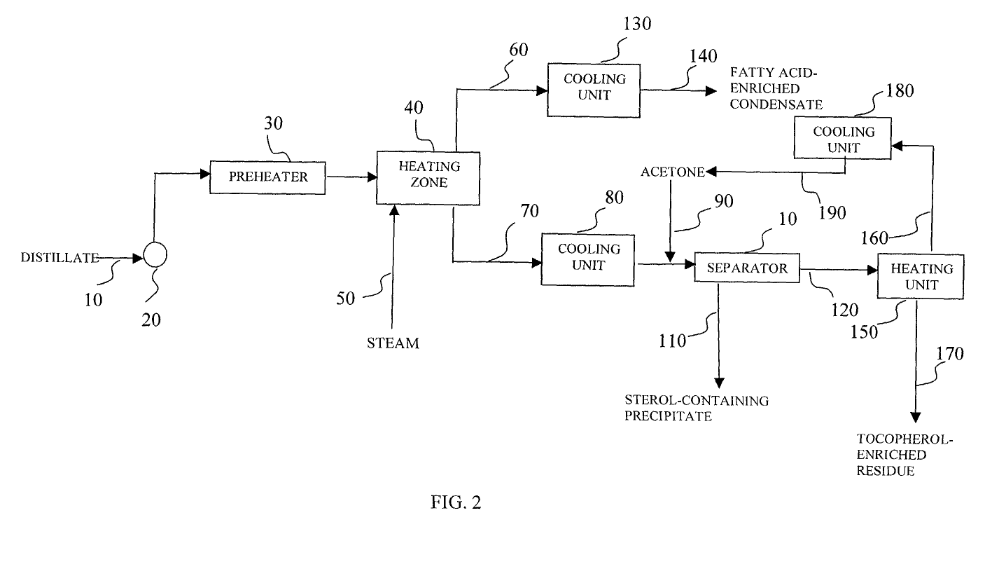

FIG. 2 illustrates another process suitable for carrying out the methods of the invention. The method illustrated in FIG. 2 again generally begins by introducing a distillate 10 comprising sterols, tocopherols, and fatty acids into a heating zone 40 operating at a pressure of less than about 10 mm Hg and at a temperature of less than about 480° F. As described above, heating zone 40 can comprise any equipment of sufficient volume and capable of operating at reduced pressure and elevated temperature. As described above, reduced pressure can be generated by any convenient source. Typically, heating zone 40 operates at a pressure of less than about 10 mm Hg. Preferably, heating zone 40 operates at a pressure of less than about 6 mm Hg. Most preferably, heating zone 40 operates at a pressure of less than about 4 mm Hg.

Optionally, but preferably, the distillate 10 passes through a preheater 30 before being introduced into heating zone 40. Preferably, the distillate 10 is preheated to a temperature near to the operating temperature of heating zone 40. The distillate 10 can be preheated either directly, as by mixing with a separate stream of heated distillate, or indirectly, as by a convenient means such as a heat exchanger.

Within heating zone 40, a substantial fraction of the fatty acid content of distillate 10 vaporizes, producing a first vapor phase 60 enriched in fatty acids and leaving a remaining fraction of distillate 70 enriched in sterols and tocopherols. To minimize the risk of thermal degradation that can occur at high processing temperatures, the distillate 10 remains in heating zone 40 for a time of less than about 60 minutes, and preferably less than about 30 minutes. Optionally, but preferably, the distillate 10 is contacted with a stripping gas to accelerate vaporization and/or removal of vaporized fatty acids. Steam or nitrogen is commonly employed as stripping gas. As described above, the usage rate of stripping gas will vary based on the type and flow rate of distillate, the distillate pre-deodorization history, and the dimensions of the heating zone(s). When the stripping gas is steam, it is generally used in an amount of from about 0.1 to about 5 percent by weight of distillate when the operating pressure is less than about 5 mm Hg. When the stripping gas is nitrogen, it is generally introduced at a rate of from about 0.5 to about 3 liters per minute when the operating pressure is less than about 5 mm Hg., which equates generally to a rate of from about 0.2 to about 20 pounds per hundred pounds of distillate.

Heating zone 40 operates at a temperature less than the boiling point of tocopherols and sterols at the operating temperature but greater than the boiling point of fatty acids at the operating pressure. Generally, heating zone 40 operates at a temperature of from about 375 to about 480° F. Preferably, heating zone 40 operates at a temperature of from about 400 to about 465° F. Most preferably, heating zone 40 operates at a temperature of from about 425 to about 450° F.

The remaining fraction of distillate 70 passes through a cooling unit 80 where it is cooled to a temperature below the boiling point of acetone. The remaining fraction of distillate 70 can be cooled either directly, as by mixing with a separate stream of cooled remaining fraction of distillate 70, or indirectly, as by a convenient means such as a heat exchanger.

The cooled remaining fraction of distillate 70 is then combined with acetone 90 in a ratio of from about 1.5:1 to about 0.5:1. Because the remaining fraction of distillate 70 contains less than about 5 percent by weight fatty acids, an extraction with acetone causes the acetone-miscible tocopherols to partition into a solvent phase and the acetone-immiscible sterols to precipitate. The solvent phase enriched in tocopherols 120 and the sterol-containing precipitate 110 can be segregated in separator 100. Such segregation can occur by a convenient method such as by gravitational force or by centrifugal separation. Preferably, separator 100 is a centrifuge. Generally, the solvent phase enriched in tocopherols 120 contains at least about 80 percent by weight of the amount of tocopherols originally present in the distillate 10. The sterol-containing precipitate 110 contains at least about 70 percent by weight sterols.

The solvent phase enriched in tocopherols 120 can be further processed to recover and recycle acetone for use in the extraction process. Specifically, the solvent phase enriched in tocopherols 120 can be passed through a heating unit 150 operating at a temperature above the boiling point of acetone at a selected operating pressure. Within heating unit 150, a substantial fraction of acetone is vaporized to produce a second vapor phase 160 enriched in acetone and a tocopherol-enriched residue 170. The second vapor phase 160 in turn can be passed through a cooling unit 180 and cooled directly or indirectly to produce a condensate 190 enriched in acetone, which can then be recycled for use in the extraction process.

The first vapor phase 60 can be passed through a cooling unit 130 to produce a condensate 140 enriched in fatty acids. The first vapor phase 60 can be cooled either directly, as by mixing with a separate stream of cooled condensate enriched in fatty acids, or indirectly, as by a convenient means such as a heat exchanger. Generally, the condensate 140 enriched in fatty acids contains at least about 70 percent by weight fatty acids.

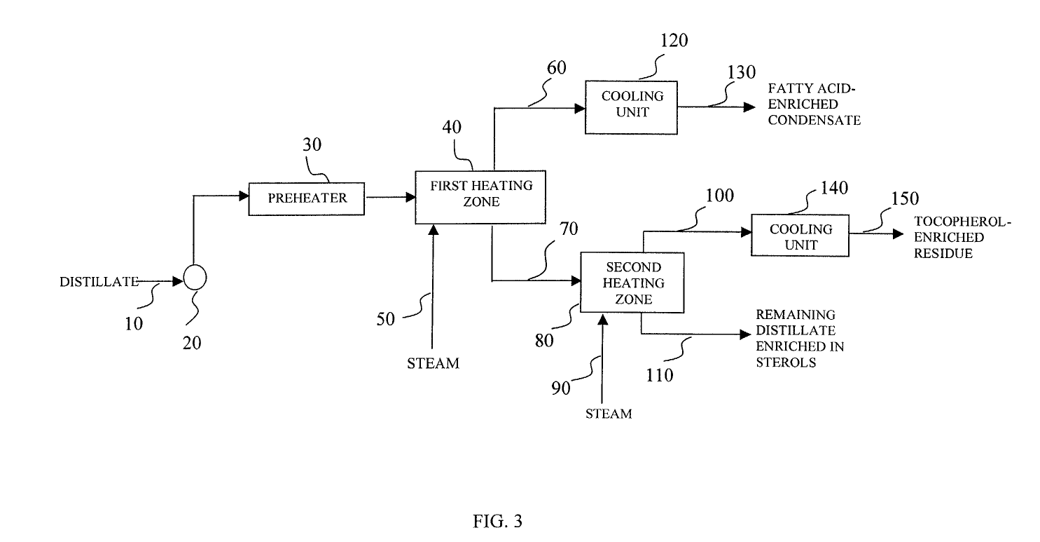

FIG. 3 illustrates yet another process suitable for carrying out the methods of the invention. The method illustrated in FIG. 3 again generally begins by introducing a distillate 10 comprising sterols, tocopherols, and fatty acids into a first heating zone 40 operating at a pressure of less than about 10 mm Hg and at a temperature of less than about 480° F. As described above, first heating zone 40 can comprise any equipment of sufficient volume and capable of operating at reduced pressure and elevated temperature. As described above, reduced pressure can be generated by any convenient source. Typically, first heating zone 40 will operate at a pressure of less than about 10 mm Hg. Preferably, first heating zone 40 operates at a pressure of less than about 6 mm Hg. Most preferably, first heating zone 40 operates at a pressure of less than about 4 mm Hg.

Optionally, but preferably, the distillate 10 passes through a preheater 30 before being introduced into first heating zone 40. Preferably, the distillate 10 is preheated to a temperature near to the operating temperature of first heating zone 40. The distillate 10 can be preheated either directly, as by mixing with a separate stream of heated distillate, or indirectly, as by a convenient means such as a heat exchanger.

Within first heating zone 40, a substantial fraction of the fatty acid content of distillate 10 vaporizes, producing a first vapor phase 60 enriched in fatty acids and leaving a first remaining fraction of distillate 70 enriched in sterols and tocopherols. To minimize the risk of thermal degradation that can occur at high processing temperatures, the distillate 10 remains in first heating zone 40 for a time of less than about 60 minutes, and preferably less than about 30 minutes. Optionally, but preferably, the distillate 10 is contacted with a stripping gas to accelerate vaporization and/or removal of vaporized fatty acids. Steam or nitrogen is commonly employed as stripping gas. As described above, the usage rate of stripping gas will vary based on the type and flow rate of distillate, the distillate pre-deodorization history, and the dimensions of the heating zone(s). When the stripping gas is steam, it is generally used in an amount of from about 0.1 to about 5 percent by weight of distillate when the operating pressure is less than about 5 mm Hg. When the stripping gas is nitrogen, it is generally introduced at a rate of from about 0.5 to about 3 liters per minute when the operating pressure is less than about 5 mm Hg., which equates generally to a rate of from about 0.2 to about 20 pounds per hundred pounds of distillate.

First heating zone 40 operates at a temperature less than the boiling point of tocopherols and sterols at the operating temperature but greater than the boiling point of fatty acids at the operating pressure. Generally, first heating zone 40 operates at a temperature of from about 375 to about 480° F. Preferably, first heating zone 40 operates at a temperature of from about 400 to about 465° F. Most preferably, first heating zone 40 operates at a temperature of from about 425 to about 450° F.

The vapor phase 60 can be passed through a cooling unit 120 to produce a condensate 130 enriched in fatty acids. The vapor phase 60 can be cooled either directly, as by mixing with a separate stream of cooled condensate enriched in fatty acids, or indirectly, as by a convenient means such as a heat exchanger. Generally, condensate 130 comprises at least about 70 percent by weight fatty acids.

The first remaining fraction of distillate 70 is introduced into a second heating zone 80 operating at a pressure of less than about 10 mm Hg and at a temperature of from about 450 to about 525° F. Second heating zone 80 can comprise any equipment of sufficient volume and capable of operating at reduced pressure and elevated temperature. As described above, reduced pressure can be generated by any convenient source. Typically, second heating zone 80 will operate at a pressure of less than about 10 mm Hg. Preferably, second heating zone 80 operates at a pressure of less than about 6 mm Hg. Most preferably, second heating zone 80 operates at a pressure of less than about 4 mm Hg.

The first remaining fraction of distillate 70 generally remains in heating zone 80 for a time of less than about 60 minutes, and preferably less than about 30 minutes. Optionally, but preferably, the first remaining fraction of distillate 70 is contacted with a stripping gas to accelerate vaporization and/or removal of volatilized components. Steam or nitrogen is commonly employed as stripping gas. As described above, the usage rate of stripping gas will vary based on the characteristics of the first remaining fraction of distillate 70. When the stripping gas is steam, it is generally used in an amount of from about 0.1 to about 5 percent by weight of the first remaining fraction of distillate 70 when the operating pressure is less than about 5 mm Hg. When the stripping gas is nitrogen, it is generally introduced at a rate of from about 0.5 to about 3 liters per minute when the operating pressure is less than about 5 mm Hg., which equates generally to a rate of from about 0.2 to about 20 pounds per hundred pounds of first remaining fraction of distillate 70.

Second heating zone 80 operates at a temperature less than the boiling point of sterols at the operating temperature but greater than the boiling point of tocopherols at the operating pressure. Generally, second heating zone 80 operates at a temperature of from about 450 to about 525° F. Preferably, second heating zone 80 operates at a temperature of from about 455 to about 515° F. Most preferably, second heating zone 80 operates at a temperature of from about 460 to about 505° F.

Within second heating zone 80, a substantial fraction of the tocopherols contained in the first remaining fraction of distillate 70 are vaporized, producing a second vapor phase 100 enriched in tocopherols and leaving a second remaining fraction of distillate 110 enriched in sterols. The second remaining fraction of distillate 110 enriched in sterols generally comprises at least about 20 percent by weight sterols.

The second vapor phase 100 can be passed through a cooling unit 140 to produce a condensate 150 enriched in tocopherols. The second vapor phase 100 can be cooled either directly, as by mixing with a separate stream of cooled condensate enriched in tocopherols, or indirectly, as by a convenient means such as a heat exchanger. Generally, condensate 150 comprises at least about 20 percent by weight tocopherols.

All documents, e.g., patents, journal articles, and textbooks, cited above or below are hereby incorporated by reference in their entirety.

One skilled in the art will recognize that modifications may be made in the present invention without deviating from the spirit or scope of the invention. The invention is illustrated further by the following examples, which are not to be construed as limiting the invention in spirit or scope to the specific procedures or compositions described therein.

### EXAMPLE 1

A distillate obtained from the deodorization of soybean oil containing approximately 30.2 percent by weight free fatty acids, 16.6 percent by weight tocopherols, and 17.6 percent by weight sterols and having a temperature of about 150° F. was directed at a rate of 60 gallons per hour to a heating unit and heated to a temperature of 450° F., producing a vapor phase and a remaining fraction of distillate. Collecting and cooling the vapor phase produced about 20 gallons per hour of a condensate containing approximately 75 percent by weight fatty acids, 5 percent by weight tocopherols, and 2 percent by weight sterols. The remaining fraction of distillate was produced in an amount of about 40 gallons per hour and contained 4.1 percent by weight fatty acids, 21.5 percent by weight tocopherols, and 20.1 percent by weight sterols.

### EXAMPLE 2

A distillate obtained from the deodorization of soybean oil containing approximately 30.2 percent by weight fatty acids, 16.6 percent by weight tocopherols, and 17.6 percent by weight sterols and having a temperature of about 150° F. was directed at a rate of 60 gallons per hour to a heating unit and heated to a temperature of 450° F., producing a vapor phase and a remaining fraction of distillate. Collecting and cooling the vapor phase produced about 20 gallons per hour of a condensate containing 77.7 percent by weight fatty acids, 4.9 percent by weight tocopherols, and 1.7 percent by weight sterols. The remaining fraction of distillate was produced in an amount of about 40 gallons per hour and contained 0.8 percent by weight fatty acids, 20.7 percent by weight tocopherols, and 17.1 percent by weight sterols.

### EXAMPLE 3

The remaining fraction of distillate of Example 2 was cooled to ambient temperature and combined with acetone in a ratio of 1:1. The resulting mixture was centrifuged to produce a sterol-containing precipitate and solvent phase enriched in tocopherols. Acetone was vaporized from the solvent phase, producing a tocopherol-enriched residue. The sterol-containing precipitate contained approximately 1.57 percent by weight fatty acids, 6.29 percent by weight tocopherols, and 76.46 percent by weight sterols. The tocopherol-enriched residue contained 11.31 percent by weight fatty acids, 42.97 percent by weight tocopherols, and 18.87 percent by weight sterols.

### EXAMPLE 4

Thirty pounds of a distillate obtained from the deodorization of soybean oil containing approximately 38.4 percent by weight fatty acids, 15.5 percent by weight tocopherols, and 17.5 percent by weight sterols was heated to a temperature of 437° F. and introduced into a deodorizer operating at a temperature of 440° F. and a pressure of about 3 mm Hg. Nitrogen stripping gas was continuously passed through the distillate in the deodorizer at a rate of about 1 liter per minute. The distillate was deodorized at 440° F. for a time of 45 minutes, producing 11 pounds of a first vapor phase, which was collected and cooled to form a first condensate, and 19 pounds of a first remaining fraction of distillate. The first condensate contained 73.2 percent by weight fatty acids, 4.6 percent by weight tocopherols, and 2.1 percent by weight sterols. The first remaining fraction of distillate contained 6.3 percent by weight fatty acids, 20.2 percent by weight tocopherols, and 12.9 percent by weight sterols.

The first remaining fraction of distillate in the deodorizer was heated to a temperature of 475° F. and then deodorized for 120 minutes in the presence of nitrogen and at a pressure of about 2 mm Hg, producing 4.5 pounds of a second vapor phase, which was collected and cooled to form a second condensate, and 14 pounds of a second remaining fraction of distillate. The second condensate contained 31.1 percent by weight fatty acids, 32.5 percent by weight tocopherols, and 10.4 percent by weight sterols. The second remaining fraction of distillate contained 0.15 percent by weight fatty acids, 35.5 percent by weight tocopherols, and 27.1 percent by weight sterols.

The second remaining fraction of distillate in the deodorizer was heated to a temperature of 500° F. and then deodorized for 200 minutes in the presence of nitrogen and at a pressure of about 3 mm Hg, producing 4.2 pounds of a third vapor phase, which was collected and cooled to form a third condensate, and 8.5 pounds of a third remaining fraction of distillate. The third condensate contained 10.5 percent by weight fatty acids, 41.3 percent by weight tocopherols, and 22.7 percent by weight sterols. The third remaining fraction of distillate contained 0.11 percent by weight fatty acids, 2.9 percent by weight tocopherols, and 5.7 percent by weight sterols.

### EXAMPLE 5

Forty-three pounds of the same distillate used in Example 4 was heated to a temperature of 423° F. and introduced into a deodorizer operating at a temperature of 430° F. and a pressure of about 2.3 mm Hg. Nitrogen stripping gas was continuously passed through the distillate in the deodorizer at a rate of about 1 liter per minute. The distillate was deodorized at 430° F. for a time of 240 minutes, producing 15 pounds of a first vapor phase, which was collected and cooled to form a first condensate, and 28 pounds of a first remaining fraction of distillate. The first condensate contained 74 percent by weight fatty acids, 4.7 percent by weight tocopherols, and 1.9 percent by weight sterols. The first remaining fraction of distillate contained 3.5 percent by weight fatty acids, 20.7 percent by weight tocopherols, and 9.1 percent by weight sterols.

The first remaining fraction of distillate in the deodorizer was heated to a temperature of 485° F. and then deodorized for 180 minutes in the presence of nitrogen and at a pressure of 3 mm Hg minimum (to keep from approaching the sterol vapor pressure at the operating temperature i.e. to prevent sterols from volatilizing), producing 5.0 pounds of a second vapor phase, which was collected and cooled to form a second condensate, and 21.5 pounds of a second remaining fraction of distillate. The second condensate contained 26.4 percent by weight fatty acids, 37.4 percent by weight tocopherols, and 7.7 percent by weight sterols. The second remaining fraction of distillate contained 0.15 percent by weight fatty acids, 16.4 percent by weight tocopherols, and 8.1 percent by weight sterols.

The second remaining fraction of distillate in the deodorizer was heated to a temperature of 500° F. and then deodorized for 180 minutes in the presence of nitrogen and at a pressure of 3 mm Hg minimum, producing 2.5 pounds of a third vapor phase, which was collected and cooled to form a third condensate, and 18.5 pounds of a third remaining fraction of distillate. The third condensate contained 14 percent by weight fatty acids, 48.3 percent by weight tocopherols, and 12.4 percent by weight sterols. The third remaining fraction of distillate contained 0.07 percent by weight fatty acids, 11.6 percent by weight tocopherols, and 6.9 percent by weight sterols.

The invention and the manner and process of making and using it, are now described in such full, clear, concise and exact terms as to enable any person skilled in the art to which it pertains, to make and use the same. Although the foregoing describes preferred embodiments of the present invention, modifications may be made therein without departing from the spirit or scope of the present invention as set forth in the claims. To particularly point out and distinctly claim the subject matter regarded as invention, the following claims conclude this specification.

What we claim is:

1. A process for isolating components from a distillate, comprising:
(a) introducing a distillate comprising sterols, tocopherols, and fatty acids into a heating zone operating at a pressure of less than about 10 mm Hg and at a temperature of less than about 480° F.;
(b) vaporizing a substantial fraction of the fatty acids to produce a vapor phase, leaving a remaining fraction of distillate enriched in sterols and tocopherols; and
(c) cooling the vapor phase to form a condensate enriched in fatty acids.

2. The process according to claim 1, wherein step (b) vaporizing occurs in the presence of a stripping gas.

3. The process according to claim 2, wherein the stripping gas is selected from the group consisting of steam, nitrogen, and mixtures thereof.

4. The process according to claim 1, wherein the distillate is preheated prior to being introduced into the heating zone.

5. The process according to claim 1, wherein the heating zone operates at a temperature of from about 375 to about 475° F.

6. A process for isolating components from a distillate, comprising:
(a) introducing a distillate comprising sterols, tocopherols, and fatty acids into a heating zone operating at a pressure of less than about 10 mm Hg and at a temperature of less than about 480° F.;
(b) vaporizing a substantial fraction of the fatty acids to produce a first vapor phase, leaving a remaining fraction of distillate enriched in sterols and tocopherols;
(c) cooling the remaining fraction of distillate;
(d) combining acetone and the remaining fraction of distillate to produce a solvent phase enriched in tocopherols and a precipitate enriched in sterols; and
(e) separating the solvent phase and the precipitate.

7. The process according to claim 6, wherein step (b) vaporizing occurs in the presence of a stripping gas.

8. The process according to claim 7, wherein the stripping gas is selected from the group consisting of steam, nitrogen, and mixtures thereof.

9. The process according to claim 6, wherein the distillate is preheated prior to being introduced into the heating zone.

10. The process according to claim 6, wherein step (e) separating occurs by centrifugation.

11. The process according to claim 6, further comprising step (f) cooling the first vapor phase to produce a condensate enriched in fatty acids.

12. The process according to claim 6, further comprising step (f) vaporizing a substantial fraction of the acetone from the solvent phase to produce a second vapor phase, leaving a residue enriched in tocopherols.

13. The process according to claim 11, further comprising step (g) vaporizing a substantial fraction of the acetone from the solvent phase to produce a second vapor phase, leaving a residue enriched in tocopherols.

14. The process according to claim 12, further comprising step (g) cooling the second vapor phase to produce a condensate enriched in acetone.

15. The process according to claim 13, further comprising step (h) cooling the second vapor phase to produce a condensate enriched in acetone.

16. The process according to claim 6, wherein the heating zone operates at a temperature of from about 375 to about 480° F.

17. The process according to claim 11, wherein the heating zone operates at a temperature of from about 375 to about 480° F.

18. The process according to claim 12, wherein the heating zone operates at a temperature of from about 375 to about 480° F.

19. The process according to claim 13, wherein the heating zone operates at a temperature of from about 375 to about 480° F.

20. A process for isolating components from a distillate, comprising:
(a) introducing a distillate comprising sterols, tocopherols, and fatty acids into a first heating zone operating at a pressure of less than about 10 mm Hg and at a temperature of less than about 480° F.;
(b) vaporizing a substantial fraction of the fatty acids to form a first vapor phase, leaving a first remaining fraction of distillate enriched in sterols and tocopherols;
(c) introducing the first remaining fraction of distillate into a second heating zone operating at a pressure of less than about 10 mm Hg and at a temperature of from about 450° F. to about 525° F.; and
(d) vaporizing a substantial fraction of the tocopherols to produce a second vapor phase, leaving a second remaining fraction of distillate enriched in sterols.

21. The process according to claim 20, wherein step (b) occurs in the presence of a stripping gas.

22. The process according to claim 21, wherein the stripping gas is selected from the group consisting of steam, nitrogen, and mixtures thereof.

23. The process according to claim 20, wherein the distillate is preheated prior to being introduced into the first heating zone.

24. The process according to claim 20, wherein step (d) vaporizing occurs in the presence of a stripping gas.

25. The process according to claim 24, wherein the stripping gas is selected from the group consisting of steam, nitrogen, and mixtures thereof.

26. The process according to claim 21, wherein step (d) vaporizing occurs in the presence of a stripping gas.

27. The process according to claim 26, wherein the stripping gas is selected from the group consisting of steam, nitrogen, and mixtures thereof.

28. The process according to claim 20, further comprising step (e) cooling the first vapor phase to produce a condensate enriched in fatty acids.

29. The process according to claim 20, further comprising step (e) cooling the second vapor phase to produce a condensate enriched in tocopherols.

30. The process according to claim 20, further comprising:
(e) cooling the first vapor phase to produce a first condensate enriched in fatty acids; and
(f) cooling the second vapor phase to produce a second condensate enriched in tocopherols.

31. The process according to claim 20, wherein the first heating zone operates at a temperature of from about 375 to about 480° F. and the second heating zone operates at a temperature of from about 470 to about 510° F.

32. The process according to claim 28, wherein the first heating zone operates at a temperature of from about 375 to about 480° F. and the second heating zone operates at a temperature of from about 470 to about 510° F.

33. The process according to claim 29, wherein the first heating zone operates at a temperature of from about 375 to about 480° F. and the second heating zone operates at a temperature of from about 470 to about 510° F.

34. The process according to claim 30, wherein the first heating zone operates at a temperature of from about 375 to about 480° F. and the second heating zone operates at a temperature of from about 470 to about 510° F.

35. The process according to claim 20, wherein the first heating zone and the second heating zone are located within a vessel having at least two heating zones.

36. The process according to claim 35, wherein the vessel is a deodorizer.
